(19) United States
(12) Patent Application Publication
(10) Pub. No.: US 2002/0018831 A1
(43) Pub. Date: Feb. 14, 2002

(54) Process for the continuous isolation of active proteins

(76) Inventors: Philippe Warnery, Savigny (CH); Marc Cedric Daury, Epalinges (CH); Marcel Alexandre Juillerat, Lausanne (CH); Simon Crelier, Savigny (CH)

Correspondence Address: WINSTON & STRAWN, 200 PARK AVENUE, NEW YORK, NY, 10166-4193, US

(21) Appl. No.: 09/859,315

(22) Filed: May 17, 2001

Related U.S. Application Data
(63) Continuation of application No. PCT/EP99/O8699, filed on Nov. 10, 1999.

(30) Foreign Application Priority Data
Nov. 20, 1998 (EP) 98203876.2

(51) Int. Cl. A23C 9/12; A23L 1/28; C12N 9/98

(52) U.S. Cl. 426/63; 435/187

(57) ABSTRACT

A process for the isolation of active proteins from plant material or from fermentation media, wherein the active proteins contained in an enzymatic solution extracted from the plant material or from the fermentation media are precipitated in an appropriate organic solvent, continuously and in a single step in a specific reactor, the conditions in the reactor being adjusted so as to obtain a precipitate of nondenatured proteins. The precipitate is then passed through a maturation step before being continuously separated.

### CROSS-REFERENCE TO RELATED APPLICATION

This application is a continuation of PCT Application No. PCT/EP99/08699, filed Nov. 10, 1999, the disclosure of which is incorporated herein by express reference thereto.

### FIELD OF INVENTION

The subject of the present invention is a process for the continuous isolation of active proteins, and in particular, of enzymes from plants or from fermentation media, and the device for their extraction.

### BACKGROUND OF THE INVENTION

Enzymes play a major role in the biogenesis of the flavors of fresh foods. The synthesis processes that the enzymes catalyze confer on the food its taste and its characteristic odor. Unfortunately, during packaging of the foods, these compounds are often lost or are thermally degraded, and the enzymes synthesizing them are inactivated.

In industry, fresh products lose their taste and their odor. This is mainly due to the treatment inflicted in order to arrive at a stable and hygienically impeccable product. The very volatile molecules responsible for the odor disappear first, those responsible for the fresh taste are impaired, and the enzymes are inactivated. The food can then be preserved more easily, but lacks real taste.

To solve this problem, synthetically produced taste enhancers are introduced. This hardly “natural” means tends to cause novel means of restoring to these products their taste to be sought. Such is the case, in particular, of products based on plants such as fruit and vegetables.

One method, proposed in U.S. Pat. No. 2,924,521 to Hewitt et al., relates to the extraction of the enzymes from the fresh plant material, and then the regeneration of the natural flavor of the food products by reintroducing the corresponding enzymes at the end of the process. The plant enzymes are extracted and precipitated several times with cold acetone. Such a process of intermittent (batch) extraction is slow, and the conditions are therefore not very reproducible, causing low productivity.

The effective isolation of the endogenous enzymes is a key step in the process of restoring taste. Numerous documents describe such processes for the extraction or isolation of endogenous plant enzymes.

For example, U.S. Pat. No. 4,728,613 to Brewer et al. describes a process for enriching the enzyme present in one of the 2 phases of an insoluble oil-water mixture. The enzyme should still be isolated, which is not described by Brewer. This process is slow and tedious since is requires several steps. Furthermore, it allows only a small yield, which is incompatible with industrial use, since a portion of the activity of the enzyme is lost at each step.

Such processes do not allow continuous isolation of the plant enzymes and, the yield and the activity of the enzymes obtained are generally very low. Thus, it is desired to use a process for the continuous isolation of active proteins, particularly at higher yields.

### SUMMARY OF THE INVENTION

To this effect, in the process for the isolation of “active” proteins from plant material or from fermentation medium according to the present invention, there are precipitated continuously and in a single step, in an appropriate organic solvent, the active proteins contained in an enzymatic solution extracted from the plant material or from the fermentation medium, in a specific reactor, the proteins contained in a solution based on the juice of plant material, the conditions in the reactor being set so as to obtain a precipitate of nondenatured proteins, the precipitate is then separated continuously.

The contact time, the rate of stirring, the temperature, and the quantity of solvent determine the quality and the quantity of protein precipitate. These parameters are therefore adjusted so as to obtain a precipitate of nondenatured proteins, as will be readily determined by one of ordinary skill in the art, particularly with respect to this application. Thus, in the case of enzymes, “active” molecules are obtained.

A step of maturation of the protein precipitate may be applied after the precipitation in the reactor, so as to increase the size of its constituent particles. It may be obtained, for example, by mixing using a vertical turbine in a continuous reactor, or by means of a static mixer.

The process according to the invention makes it possible to obtain a nondenatured, and therefore active, protein extract, in particular enzymes. Using such a process, the yield of extraction as well as the activity of the enzymes are much higher than that which can be obtained by conventional processes or batch processes.

Another subject of the invention is a reactor that allows the continuous isolation of numerous active proteins. It is possible, for example, to extract more pectin methylesterase (PME) and peroxidase (POX) activity and a higher quantity of proteins (see Table 1 below). It consists of an angle-shaped, and preferably T-shaped, cell. The reaction conditions can be easily adjusted and permit optimization of the process for each type of protein.

This reactor also has the advantage of having a simple geometry compared with other reactors, making it very easy to operate and to clean.

The invention finally relates to the nondenatured protein extract thus obtained, and its use for regenerating the flavors and tastes of various food products such as, for example, soups and other vegetable-based products, baby foods.

This process can also be applied in the field of biotechnology for “downstream processing”, i.e., the separation of an enzyme produced by micro-organisms in, for example, a biofermenter.

### DETAILED DESCRIPTION OF THE INVENTION

The expression “fresh taste or flavor” as used herein refers to the flavor and taste of fresh tomato, that is to say the green, acid, and light notes that are not found in, for example, industrial tomato juice.

The term “active” proteins as used herein designates the enzymes present in the tomato that are partially responsible for the taste and the odor that are known to be nondenatured. These active proteins are, for example, enzymes such as peroxidase (POX), acid phosphatase (AP), pectin methylesterase (PME), and alcohol dehydrogenase (ADH).

To carry out the present process, any plant material may be used, such as fruits and/or vegetables, i.e., any edible plant, whether it is, for example, a seed, root, tuber, stem, leaf, flower, or fruit. Nevertheless, plants are preferably used for which it is desired to enhance the natural fresh taste. Plants whose natural taste may be unpleasant or whose cooked taste is sought after will therefore be particularly avoided, especially, for example, asparagus, garden pea, soybean, potato, cereals, sea buckthorn berry, and medlars.

Among the preferred plants, for example, more particular leaves, in particular leek, fennel and cabbage; stems, in particular rhubarb and broccoli; certain roots, in particular carrot, onion, radish, celery and beet; tubers, in particular cassava; and fruit, in particular tomato, courgette, eggplant, banana, apple, apricot, melon, watermelon, pear, plum, peach, cherry, kiwi and mirabelle plum, may be used.

Edible higher mushrooms that may be considered to be included among plants may be used. Preferable mushrooms include Agaricus bisporus, Pleurotus ostreatus, Boletus edulis, or Lentinus edodes.

Advantageously, industrial plant “waste” such as, for example, the skins, leaves and branches, may be used in the process of the invention.

The plant material may be prepared in the form of juice, and then treated so that the solution contains as much enzyme as possible. This initial extraction step includes solubilizing the maximum quantity of enzymes before the step for the actual isolation in a specific reactor. The plant material may be homogenized, and then the pH of the homogenate brought to 5 to 8.5, preferably 7. Salt, for example sodium chloride, may then be added. The total salt concentration may be from 0.25 to 1M, preferably 0.5M. The insoluble portions may then be removed by, for example, centrifugation. The optimum conditions for each enzyme are shown in Table 2. The supernatant thus obtained may be frozen or directly treated in the reactor so as to isolate the active proteins therefrom. The yield of extraction of the enzymes may be, for example, from 50% and 100% for tomato.

The solution containing the enzymes to be isolated is thus continuously introduced into a reactor consisting of a cell, two “inlet” (enzymatic solution and solvent) branches, and an “outlet” branch (for the enzyme isolate in the form of a precipitate), the latter preferably forming an angle of 90° relative to the inlets, that is a T-shaped cell. Other angles may also be used. The mixing of the solution containing the enzymes to be isolated with the organic solvent is then carried out in the reactor.

The solvent is preferably chosen from alcohols, in particular ethanol, or any other derived organic solvent. The solvent is directly injected into the cell, through one of the inlet branches of the reactor cell. Alcohol is preferably used such that its final concentration is from 40% to 95% by mass, preferably 80%.

The conditions in the reactor are adjusted so as to obtain a precipitate of nondenatured proteins. The contact time and the cooling temperature are preferably chosen so that the internal temperature of the mixture remains low, such that the enzymes are not denatured. To this effect, temperatures of, for example, −15° C. to +18° C., and preferably about 0° C., will be used in the reactor. The protein precipitate is preferably in contact with the solvent, after passage into the reactor, for 0 to 30 minutes, and preferably for 30 seconds.

The optimum conditions for isolating various enzymes are preferably a final temperature of 0° C. in the T-shaped reactor, a final ethanol concentration of 80% and a contact time for the precipitate with the solvent of about 30 seconds.

The suspension of precipitate is continuously discharged through the outlet which preferably forms an angle of 90° relative to the inlet for the enzymatic solution and the solvent. The size of the particles of the suspension may vary from 1 to 2 microns.

To complete the isolation after the precipitation in the reactor, the suspension of precipitate may be subjected to a maturation step in order to increase the size of the particles of the suspension. A continuously-stirred tank-type reactor or a static mixer may be used for this step. The conditions of duration and rate of mixing are preferably adjusted so as to obtain particles or aggregates of sufficient size. Thus, the suspension of precipitate may be either, for example, mixed at a temperature close to 4° C. in a stirred tank provided with a vertical helix at a rate of 100 rpm to 400 rpm (corresponding to a Reynolds (Re) number of 1175 to 4700) for 10 to 60 seconds, preferably at 300 rpm (Re of about 3500) for 20 seconds, or put through static mixers at 4° C. for 30 seconds. The precipitate obtained after maturation includes aggregates whose size may be up to 500 microns on average. The precipitate is then separated continuously.

The continuous separation of the protein precipitate is preferably obtained by simple centrifugation. The pellet is recovered and then stored. The supernatant may be either eliminated or treated in a distillation column and the ethanol thus recovered recycled into the process. The enzymatic extract thus obtained may, for example, then be directly frozen without addition of water, or freeze-dried.

Between 25 and 100% of the activity of the enzymes may be preserved, depending on the fragility of the enzyme (see Tables 1 to 3). For example, for tomato, 25% to 50% of the activity of PME, from 80% to 100% of the activity of POX, 70% to 100% of the activity of ADH, and 80 to 100% of AP may be recovered. Furthermore, the protein isolation yield may be from 50% to 95%.

The process according to the invention also makes it possible to obtain a yield of isolation of the enzymes greater than what is normally obtained by conventional processes or batch processes (see Table 4).

According to another aspect of the invention, the reactor is, for example, preferably a T-shaped cell made of Plexiglas®, with no mixer. The shape of the reactor is such that there are as few dead spaces as possible. The inlet streams are preferably at the base of the mixture volume with a diameter of the inlet tubes identical, preferably about 1.5 mm, and the outlet stream, perpendicular to the other two, being at the top. The diameter of the outlet tube is preferably ⅓ larger than those of the inlet streams. For example, the diameter of the outlet tubes may be 2 mm, when the inlet tubes are 1.5 mm. These diameters may vary according to the throughputs to be passed through the reactor, but they should preferably be chosen so as to ensure a speed of the stream of the source of enzymes at the time of contact of, for example, about 5 cm/s to 20 cm/s, and preferably of about 11 cm/s, so as to allow good mixing while avoiding possible denaturation of the enzymes.

The present invention is capable of treating, for example, up to 14 tons of tomatoes per day, with sizes of the branches of the reactor on the order of 4 cm for the inlets and about 5.2 cm for the outlet.

Another aspect of the invention relates to the use of the enzymes or of the endogenous proteins isolated according to the invention for the preparation of cosmetic or food products.

The enzymes may also be used to regenerate the flavor or the taste of preparations, such as soups, baby foods, vegetable purees or juices, or prepared meat products. The process proves particularly effective for the extraction of “active agents” from, for example, tomatoes, carrots, onions. If tomato, for example, is chosen as plant material, the enzymes continuously extracted by the process according to the invention may be used in tomato juices, tomato puree, all the tomato-based deep-frozen and fresh products such as, for example, pizzas and lasagnes.

The process of the invention may also be applicable to the field of biotechnology for “downstream processing,” i.e., the separation of an enzyme produced by microorganisms in, for example, a biofermenter.

### EXAMPLES

The present invention is described in detail with the aid of the examples that follow. These examples are given by way of illustration of the subject of the invention and do not constitute in any manner a limitation thereto. The percentages given therein are by weight unless otherwise stated.

### Example 1

### Isolation of Enzymes from Tomato

Tomatoes were washed and then processed into juice. The juice was then treated by a first extraction step so as to solubilize the maximum quantity of enzymes before the actual isolation step in the specific reactor. Thus, the plant material was homogenized and then the pH of the homogenate was brought to 7 by addition of a sodium hydroxide solution. NaCl was then added so that the final salt concentration is 0.5M. The insoluble parts were then removed by centrifugation. The supernatant thus obtained can be frozen or directly treated in the reactor so as to isolate the active proteins therefrom.

The solution containing the enzymes to be isolated was then introduced through one of the inlet branches of the T-shaped reactor. The ethanol was directly injected into the cell through the other inlet branch of the reactor cell. The final ethanol concentration was 80%.

The precipitate of nondenatured proteins obtained was continuously discharged through the T-branch placed at 90° relative to the inlet branches. The temperature of the mixture was about 0° C. The suspension of precipitate was then vigorously mixed with a vertical turbine for some 20 seconds (contact time). The precipitate was then continuously separated by centrifugation. The pellet was recovered and then stored. The supernatant may be either removed, or treated in a distillation column, and the ethanol thus recovered recycled to the process.

The enzymatic extract may be either directly frozen (without addition of water), or freeze-dried. The enzymes thus isolated by the process according to the invention had an activity yield considerably higher than that which could be obtained by, for example, traditional batch processes.

The process is easy to carry out, and is, therefore, particularly effective for the continuous isolation of active proteins. Table 1 below gives the yields of activities recovered (in %) for pectin methylesterase (PME), peroxidase (POX), alcohol dehydrogenase (ADH), and acid phosphatase (AP).

TABLE 1
Yields of activity recovered for various enzymes present in tomato.

| Enzymes | Activity Yield (%) |
|---|---|
| PME | 25-50 |
| POX | 80-100 |
| ADH | 70-100 |
| AP | 80-100 |

### Example 2

### Isolation of Enzymes from Carrot

The carrots were prepared as described in Example 1. The conditions for continuous precipitation were 80% ethanol and a final temperature of 0° C.

Table 2 below gives the yields of activity recovered for alcohol dehydrogenase (ADH) and acid phosphatase (AP).

TABLE 2
Yields of activity recovered for various enzymes present in carrot.

| Enzymes | Activity Yield (%) |
|---|---|
| ADH | 70.7 |
| AP | 90.2 |

### Example 3

### Isolation of Enzymes from Onion

Onions were also prepared as in Example 1. The continuous precipitation conditions were 80% ethanol and a final temperature of 0° C.

Table 3 below gives, for example, the yields of activity recovered for cysteine sulfoxide lyase (CSL) and for peroxidase (POX).

TABLE 3
Yields of activity recovered for various enzymes present in onion.

| Enzymes | Activity Yield (%) |
|---|---|
| CSL | 73-100 |
| POX | 100 |

### Example 4

### Other Processes for Batch Isolation of Enzymes

a) Process for Batch Precipitation with Ethanol

A batch reactor and a vertical helix were used. Ethanol at 94% w/w was added to the tomato extract (80 g, at 4° C.) initially present in the reactor, until the desired concentration was obtained and then the stirring of the mixture was continued.

The recovery of pectin methylesterase activity was then measured. For a final concentration of 77%, this value varies from 0 to 20% according to the temperature of the mixture. Furthermore, this enzyme is irreversibly denatured if it remains in contact with the ethanol for an excessively long period. It should also be noted that no activity can be measured in the supernatant.

b) Process for Precipitation with Polyethylene Glycol (PEG)

The tomato extract was mixed with a 33.3% PEG 8000 solution in a batch reactor, cooled to 4° C. A slight precipitation appeared from a final PEG concentration of 12.35%.

The solution was then centrifuged at 4° C. for 10 minutes at 2000 g. The pellet was recovered and dissolved in water (as for the precipitations with ethanol) before measuring the enzymatic activities present after precipitation.

The results are given in Table 4 below.

TABLE 4
Yields of activity recovered for various enzymes present in tomato by
batch processes with PEG 8000.

| | Activity Yield | Activity Yield |
|---|---|---|
| Enzymes | with 12.35% PEG (%) | with 20% PEG (%) |
| PME | 2.4 | 16.4 |
| POX | 0 | 2 |
| ADH | 0 | 0 |
| AP | 18.8 | 72.3 |

The yields of enzymatic activity were much lower when prepared by such batch processes. In the case of the process for precipitation with PEG, the final solution was viscous and difficult to centrifuge and handle (pump). Moreover, from 25% PEG, no pellet could be obtained after centrifugation.

### Example 5

### Optimization of the Conditions for Initial Extraction of the Enzymes

To optimize the initial extraction of various enzymes from a tomato juice, the recovered activity of the enzymes was measured for pH values of from 4.2 (neutral pH) to 8.5 and for increasing NaCl concentrations (from 0 to 6%).

4.5 kg of tomatoes were washed and then processed into juice. The juice was divided into 4 fractions of 1.1 kg, of initial pH 4.2 (that of tomato). The pH of fractions 2, 3, and 4 were adjusted to 5.5, 7, and 8.5, respectively, by addition of 10, 14, and 18 g of a 20% sodium hydroxide solution. Each fraction was then distributed into vessels containing increasing quantities of NaCl: 0, 2.25, 4.5, 6.75, and 9 g, corresponding to concentrations by mass of: 0, 1.48, 2.91, 4.31, and 5.66%.

After incubating for 45 minutes at 4° C., with stirring, the various enzymatic solutions were centrifuged at 2000 g for 10 minutes. The supernatants were recovered and then there were determined for each solution the quantity of nitrogen derived from proteins and the activities of the following enzymes: peroxidase, acid phosphatase, lipoxygenase, alcohol dehydrogenase, pectin methylesterase, and polygalacturonase.

The results are presented below in Table 5. They show the optimum conditions for extraction of each type of enzyme. The conditions that gave the most satisfactory overall results for the extraction were a pH of 7 and a salt concentration of 3%.

TABLE 5
Optimum conditions for the extraction of
various tomato enzymes and proteins.

| Nitrogen/enzymes | NaCl (%) | PH |
|---|---|---|
| Nitrogen | 1.5 to 6 | 8.5 |
| Peroxidase (POX) | 0 to 6 | 4.2 |
| Lipoxygenase (LOX) | 0 to 6 | 8.5 |
| Alcohol dehydrogenase (ADH) | 0 to 6 | 7 |
| Pectin methylesterase (PME) | 1.5 to 6 | 4.2 to 8.5 |
| Polygalacturonase (PG) | 3 to 6 | 4.2 |
| Acid phosphatase (AP) | 1.5 to 6 | 5.5 or 7 |

### Example 6

### Optimization of the Conditions for Isolating Various Enzymes

To optimize the isolation process, the various precipitation parameters (final ethanol concentration, temperature in the reactor, stirring or precipitate maturation time) were varied and the recoveries of enzyme activity measured after precipitation. The optimum precipitation conditions for each enzyme are described in the table below.

TABLE 6
Optimum conditions for precipitation of a few enzymes.

| Enzymes | (Ethanol)_{f} (%) | T_{f} of the mixture (° C.) | Stirring (rpm) | Contact time |
|---|---|---|---|---|
| Peroxidase | 70-80 | −13 to +18° C. | Insensitive | Insensitive |
| Acid phosphatase | >60 | −13 to −7° C. | Insensitive | Insensitive |
| Pectin methylesterase | 90 | −13° C. | <200 | Sensitive |
| Alcohol dehydrogenase | >80 | −11 to 0° C. | <200 | Insensitive |

General optimum conditions: 80% ethanol, 0° C., without stirring, and a contact time of the precipitate with the solvent of about 30 seconds (with vigorous stirring).

### Example 7

### Optimization of the Maturation Conditions for the Precipitate

To optimize the isolation process, the suspension of precipitate obtained at the outlet of the reactor was subjected to stirring at a temperature of 4° C. in a stirred tank provided with a vertical turbine. The size of the particles was measured for mixing rates of between 100 and 400 rpm (Re of 1175 to 4700).

TABLE 7
Median size of the particles of the precipitate (in μm) as a
function of the mixing rate and time during the maturation step.

| | Time(s) | | | | | | | | | |
|---|---|---|---|---|---|---|---|---|---|---|
| | 0 | 10 | 20 | 30 | 45 | 60 | 120 | 240 | 360 | 600 |
| 100 rpm | 1.4 | 17.9 | 63.9 | 132.9 | 393.6 | 410.9 | 471.9 | 534.6 | 561.4 | 466.7 |
| 200 rpm | 1.4 | 116.9 | 296.0 | — | 567.6 | 369.5 | 314.4 | 253.1 | 235.5 | 213.0 |
| 300 rpm | 1.4 | 265.2 | 406.1 | 405.7 | 445.8 | 416.4 | 384.4 | 191.3 | 158.0 | 150.4 |
| 400 rpm | 1.4 | 309.3 | 380.6 | 443.6 | 424.1 | 284.5 | 159.6 | 118.5 | 111.7 | 111.3 |

The stirring time and rate had a great effect on the median size of the particles of the precipitate and their aggregation. The optimum conditions were a rate of 300 rpm for about 20 s.

### Example 8

### Comparison of the Yields for the T-shaped Reactor and a CSTR

Various trials for precipitation of several tomato juices were carried out in the T-shaped reactor, under optimum conditions, i.e., 80% ethanol and a final temperature of 0° C. These trials were compared to the case of a CSTR (continuous stirred-tank reactor) under the same conditions with a mixing rate of 180 rpm. The results are presented in Table 8 below.

TABLE 8
Enzymatic activity and mass yields for the
T-shaped reactor according to the invention and a CSTR.

| Recovery (%) | T | CSTR |
|---|---|---|
| POX | 98.4 | 92.4 |
| AP | 78.2 | 81.9 |
| PME | 32.6 | 29.7 |
| ADH | 78.3 | 86.2 |
| Protein nitrogen | 91.8 | 82.5 |

These comparative trials show the advantage of the T-shaped reactor compared with the CSTR for the isolation of certain enzymes that are more sensitive to ethanol and to the mixing conditions (PME, and the like). The overall yield (protein nitrogen) was substantially higher in the case of the T-shaped reactor, demonstrating a better isolation in this case.

### Example 9

### Regeneration of the Flavor and of the Taste

Sensory evaluations of tomato-based products treated with isolated endogenous enzymes were performed. The isolated enzymes obtained by the process as described in Example 1 were solubilized in water with 0.1M NaCl, and then mixed at various concentrations with 2 substrates: dilute tomato paste and tomato juice.

The treated and untreated samples were incubated for one hour at 37° C. The various samples were then tested by a panel, as follows:

description of the taste and of the flavor of several samples and preference of the testers;

for the triangular tests, 3 samples are prepared, of which 2 are identical;

the testers would determine the sample that appears different to them.

In the comments below, the quantity of enzymes added is given in %. For example, if 100 g of tomato paste (initially corresponding to 600 g of fresh tomatoes) are treated with 10% of enzymes, the quantity of enzymes recovered after precipitation of 60 g of fresh tomatoes was added to the product. The following observations were made:

if less than 10% of enzymes was used, no difference was noted between the treated and untreated samples;

an addition of 10 to 30% caused a pleasant taste corresponding to fresh tomato (slight acidity with light notes);

for the triangular test with 20% of enzymes, 100% recognition by the panel;

for quantities greater than 40%, the panel found that the samples thus treated have notes that were too acidic and green.

These results indicate and confirm the potential of the enzymatic precipitate obtained according to the invention for the regeneration of the taste and the flavor in various food products. Several enzymes necessary for the development of the taste and of the flavor in tomato were therefore present and active in this extract.

It is to be understood that the invention is not to be limited to the exact configuration as illustrated and described herein. Accordingly, all expedient modifications readily attainable by one of ordinary skill in the art from the disclosure set forth herein, or by routine experimentation therefrom, are deemed to be within the spirit and scope of the invention as defined by the appended claims.

What is claimed is:

1. A process for the isolation of active proteins from plant material or from fermentation media, comprising:
extracting an enzymatic solution containing the active proteins from the plant material or fermentation media;
precipitating the active proteins in an organic solvent in a reaction zone at a temperature of −15° C. to +18° C. for a sufficient time to precipitate active proteins; and
separating the active proteins from the precipitate;
wherein the proteins are precipitated continuously and in a single step in the reaction zone and the organic solvent reaches a final concentration in the reaction zone of 40% to 95%.

2. The process of claim 1, wherein the plant material comprises one or more of an edible seed, root, tuber, stem, leave, or flower from a fruit or vegetable plant.

3. The process of claim 1, wherein the precipitation time is up to 30 minutes, and the active proteins comprise one or more of peroxidase (POX), pectin methylesterase (PME), polygalacturonase (PG), alcohol dehydrogenase (ADH) or acid phosphatase (AP).

4. The process of claim 1, wherein the enzymatic solution is prepared as a homogeneous juice from the plant material or the fermentation media, the juice having a pH of 5 to 8.5.

5. The process of claim 4, wherein the juice comprises 0.25 to 1M salt.

6. The process of claim 1, further comprising maturing the precipitate before separating the active proteins therefrom, wherein the maturing step comprises mixing the precipitate for 10 seconds to 60 seconds at 100 rpm to 400 rpm to increase the size of particles forming the precipitate.

7. The process of claim 6, wherein the precipitate is in contact with the solvent for at least 30 seconds in the reaction zone and the temperature during the maturing step is 4° C.

8. The process of claim 1, wherein the organic solvent comprises alcohol.

9. The process of claim 8, wherein the alcohol comprises ethanol.

10. The process of claim 8, wherein the temperature in the reaction zone is 0° C. and the final alcohol concentration is 80%.

11. The process of claim 1, wherein 50% to 95% of the proteins are isolated.

12. The process of claim 1, wherein the plant material or fermentation media comprises at least one of leaves, stems, roots, tubers, fruit, or a combination thereof.

13. The process of claim 12, wherein the plant material or fermentation media comprises at least one of leek, fennel, cabbage, rhubarb, broccoli, carrot, onion, radish, celery, beet, cassava, tomato, courgette, eggplant, banana, apple, apricot, melon, watermelon, pear, plum, peach, cherry, kiwi, mirabelle plum, or a combination thereof.

14. An enzymatic alcoholic extract from a plant material, comprising one or more active proteins of peroxidase (POX), pectin methylesterase (PME), polygalacturonase (PG), alcohol dehydrogenase (ADH) or acid phosphatase (AP), and having recovered activities of 25% to 50% for PME, from 80% to 100% for POX, from 70% to 100% for ADH, or from 80% to 100% for AP.

15. A method for regenerating taste and flavor of a fruit or vegetable-based food product comprising adding the extract of claim 14 to the food product.

16. The method of claim 15, wherein the food product comprises soup, baby food, sauce, puree, a prepared meal, or a prepared meat.

17. A food product prepared by the method of claim 15.

18. A food product containing an enzymatic alcoholic extract according to claim 14.

19. A device for continuous isolation of active proteins, comprising:
a thermoregulable cell having a first inlet branch and a second inlet branch, the first inlet branch for a solution containing the active proteins to be isolated, the second inlet branch for an organic solvent; and
an outlet branch for a protein precipitate obtained, the inlet branches forming a defined angle relative to the outlet branch.

20. The device of claim 19, in which the outlet branch forms an angle of 90° relative to each inlet branch.
